(19) United States
(12) Patent Application Publication
(10) Pub. No.: US 2026/0193178 A1
(43) Pub. Date: Jul. 9, 2026

(54) COMPOSITION COMPRISING AT LEAST ONE SOLID ORGANIC PEROXIDE

(71) Applicant: ARKEMA FRANCE, Colombes (FR)

(72) Inventors: Alfredo DEFRANCISCI, Pierre-Benite (FR); Mathieu BAILLET, Pierre-Benite (FR)

(73) Assignee: ARKEMA FRANCE, Colombes (FR)

(21) Appl. No.: 18/868,927

(22) PCT Filed: Jul. 27, 2023
(86) PCT No.: PCT/FR2023/051194

§ 371 Date: Nov. 25, 2024

(30) Foreign Application Priority Data
Jul. 29, 2022 (FR) 2207910

(51) Int. Cl. C07C 407/00; C08F 2/40; C11D 3/39

(52) U.S. Cl. CPC C07C 407/006; C08F 2/40; C11D 3/3905; C11D 3/3947

(57) ABSTRACT

A composition including at least one solid organic peroxide, at least one polyol having a melting temperature of less than 40° C., preferably less than 30° C., more preferentially less than 20° C., at least one dispersant and at least one thickener. Also, the use of the composition as defined previously as a polymerization initiator, as a polymer modifier, or as a bleaching agent. Also, a process for preparing such a composition.

The present invention relates to a composition comprising at least one solid organic peroxide, at least one polyol having a melting temperature of less than 40° C., preferably less than 30° C., more preferentially less than 20° C., at least one dispersant and at least one thickener.

The invention also relates to the use of said composition as a polymerization initiator, as a polymer modifier, or as a bleaching agent.

The present invention also relates to a process for preparing such a composition.

Organic peroxides most often constitute highly unstable species since they decompose relatively easily under the action of a slight input of heat. Thus, in the event of an uncontrolled increase in their storage temperature, certain organic peroxides can undergo a self-accelerating exothermic decomposition, leading to fires and/or violent explosions. In addition, under these conditions, some of these organic peroxides notably release combustible vapors that are capable of reacting with any source of ignition, which can significantly increase, or even accelerate, the risks of violent explosion.

Such risky behavior therefore proves to be incompatible with the rules in force regarding transportation and storage of dangerous goods. Notably, it proves to be important to take appropriate precautionary measures during the storage and handling of such organic peroxides, notably by ensuring that they remain at temperatures below their self-accelerating decomposition temperature T_{SAD} to minimize the risks of fire and/or uncontrolled decomposition.

In order to overcome these various drawbacks, organic peroxides can be diluted or mixed with inert products, that is to say products that do not react in contact with the organic peroxide, called phlegmatizers. Several possibilities exist: solubilization in a liquid phlegmatizer, mixing with a solid inert phlegmatizer. The organic peroxide compositions thus obtained are suspensions or pastes. In particular, phlegmatizers make it possible to limit the effects during the uncontrolled decomposition of organic peroxides and to reduce, consequently, the various risks linked to their handling.

However, such compositions must remain stable over time, notably to avoid phase separation (decantation, clarification) between the solid organic peroxide and the phlegmatizer. Moreover, such compositions must be easy to handle and meter out for better use, notably in thermosetting resins.

Thus, one of the objectives of the present invention is notably to propose a stable and homogeneous organic peroxide-based composition that is notably intended to be used as polymerization initiators or as a polymer modifier (for example as a polymer crosslinking agent, grafting agent or rheology modifier), or as a bleaching agent, and that does not have the drawbacks described previously.

A subject of the present invention is therefore notably a composition comprising:
at least one solid organic peroxide,
at least one polyol having a melting temperature of less than 40° C., preferably less than 30° C., more preferentially less than 20° C.,
at least one dispersant, and
at least one thickener.

The composition according to the invention thus represents an organic peroxide composition that has the necessary viscosity to be easy to handle while remaining stable, whether in storage or during handling.

The present invention also relates to the use of the composition according to the invention as a polymerization initiator, in particular as a polymerization initiator of acrylic resins or of unsaturated polyester resins, preferably of acrylic resins, as a polymer modifier, in particular as a polymer crosslinking agent, grafting agent or rheology modifier, or as a bleaching agent, in particular as a laundry bleaching agent.

The present invention also relates to a process for preparing a solid organic peroxide composition, comprising a step of mixing at least one solid organic peroxide, at least one polyol having a melting temperature of less than 40° C., preferably less than 30° C., more preferentially less than 20° C., at least one dispersant and at least one thickener.

Other features and advantages of the invention will become more clearly apparent on reading the description and the examples that follow.

In the text hereinbelow, unless otherwise indicated, the limits of a range of values are included in that range.

The expression “at least one” is equivalent to the expression “one or more”.

### Composition

For the purposes of the present invention, “ambient temperature” is understood to mean a temperature ranging from 15° C. to 27° C., preferably ranging from 20° C. to 25° C.

For the purposes of the present invention, “solid organic peroxide” is understood to mean that the compound is in the solid state at ambient temperature and under atmospheric pressure (around 0.1 MPa).

Preferably, the solid organic peroxide is chosen from the group consisting of solid diacyl peroxides, solid peroxydicarbonates, solid ketone peroxides, solid peroxy esters, solid hydroperoxides, solid dialkyl peroxides, and mixtures thereof.

The solid diacyl peroxides are preferably chosen from the group consisting of dibenzoyl peroxide, di(2-methylbenzoyl) peroxide, di(methoxybenzoyl) peroxide, di(2-methoxycarbonylbenzoyl) peroxide, di(2-benzylbenzoyl) peroxide, di(2-benzylbenzoyl) peroxide, di(4-fluorobenzoyl) peroxide, di(3-chlorobenzoyl) peroxide, di(4-chlorobenzoyl) peroxide, di(2,4-dichlorobenzoyl) peroxide, dibenzoyl diperoxyadipate, benzoyl octadecanoyl peroxide, dilauroyl peroxide, dihexadecanoyl peroxide, di(chloroacetyl) peroxide, and di(3-carboxypropionyl) peroxide.

The solid peroxydicarbonates are preferably dialkyl peroxydicarbonates, notably chosen from the group consisting of dibenzyl peroxydicarbonate, dicyclohexyl peroxydicarbonate, di(cis-3,3,5-trimethylcyclohexyl) peroxydicarbonate, di(4-t-butylcyclohexyl) peroxydicarbonate, dibornyl peroxydicarbonate, di(2-phenoxyethyl) peroxydicarbonate, di-n-tridecyl peroxydicarbonate and di-n-hexadecyl peroxydicarbonate.

The solid ketone peroxides are preferably chosen from the group consisting of di(1-hydroxycyclohexyl) peroxide, 1-hydroxycyclohexyl-1-hydroperoxycyclohexyl, di(hydroperoxycyclohexyl) peroxide and 3,5-dihydroxy-3,5-dimethyl-1,2-dioxolane.

The solid peroxy esters are preferably chosen from the group consisting of di-t-butyl diperoxyterephthalate, di-t-butyl diperoxysuccinate, di-t-butyl diperoxyadipate, di-t-butyl diperoxyphthalate, peroxy-(t-butyl 3-carboxypropionate), t-butylperoxy-(3-carboxy-2-propenoate) and 2,5-dimethyl-2,5-di(benzoylperoxy) hexane.

The solid alkyl hydroperoxides are preferably chosen from the group consisting of 2,5-dimethyl-2,5-dihydroxyperoxyhexane, 2,5-dimethyl-2,5-dihydroxyperoxy-3-hexyne, 2,7-dimethyl-2,7-dihydroperoxy-3,5-octadiyne, 1,4-di(1-methyl-1-hydroxyperoxyethyl)benzene and 1,3,5-tri (1-methyl-1-hydroperoxyethyl)benzene.

The solid dialkyl peroxides are preferably chosen from the group consisting of dicumyl peroxide, 1,4-di [1-methyl-1-(tert-butylperoxy)ethyl]benzene, 1,3-di [1-methyl-1-(tert-butylperoxy)ethyl]benzene and di(isopropylcumyl) peroxide.

Preferably, the solid organic peroxide is chosen from the group consisting of solid diacyl peroxides, solid peroxydicarbonates, and mixtures thereof. Even more preferentially, the solid organic peroxide is chosen from the group consisting of solid diacyl peroxides.

Preferably, the organic peroxide is substituted or unsubstituted dibenzoyl peroxide, more preferentially unsubstituted dibenzoyl peroxide, such as the product sold under the trade name Luperox® A75 by Arkema.

The solid organic peroxide is present preferably in a content ranging from 10% to 70% by weight, preferably in a content ranging from 20% to 60% by weight, and more preferentially in a content ranging from 30% to 55% by weight, more preferentially between 40% and 50% by weight, relative to the total weight of the composition according to the invention.

The composition according to the invention comprises a polyol having a melting temperature of less than 40° C., preferably less than 30° C., more preferentially less than 20° C.

The melting temperature is determined by differential scanning calorimetry (DSC) with a temperature increase rate of 10° C./min. The melting temperature thus corresponds to the top of the endothermic melting peak obtained during the measurement.

Preferably, said polyol having a melting temperature of less than 40° C., preferably less than 30° C., more preferentially less than 20° C., is chosen from the group consisting of glycerol or diglycerol, and mixtures thereof.

Particularly preferably, said polyol is glycerol.

Preferably, said polyol is present in a content ranging from 5% to 60% by weight of the composition, preferably 10% to 50% by weight of the composition, more preferentially between 20% and 45% by weight of the composition.

The composition according to the invention further comprises at least one dispersant, which may be ionic or nonionic.

Preferably, the dispersant has an HLB of less than 14, preferably less than 13, more preferentially less than 12.

“HLB” or “HLB value” is understood to mean the hydrophilic-lipophilic balance which makes it possible to assess the solubility of a dispersant in water. Preferably, the HLB is determined according to the method proposed by Griffin (Journal of the Society of Cosmetic Chemists, 5 (4), (1954), 249-256).

If the composition comprises more than one dispersant according to the invention, then the HLB of said dispersants can be calculated from the mass ratio of the dispersants.

Preferentially, the nonionic dispersant is chosen from the group consisting of linear or nonlinear ethoxylated fatty alcohols, more preferentially nonlinear ethoxylated fatty alcohols, more preferentially ethoxylated phenols, aliphatic salts such as sodium oleate and potassium soaps of castor oil; alkylallyl sulfonates such as salts of N-acylamino acids, alkyl ether carboxylates, acylated peptides, alkyl sulfonates and sodium dodecylbenzenesulfonates; salts of sulfated esters of higher alcohols such as alkyl naphthalene sulfonates, derivatives of naphthalene sulfonates, sodium salts of β-naphthalene sulfonate formalin condensates, dialkyl sulfosuccinates, α-olefin sulfonates, N-acyl sulfonates and sodium cetyl sulfate; and macromolecular surfactants such as polyoxyethylene alkyl ether sulfates, polyoxyethylene alkylphenol ether sulfates, alkylamide sulfates, alkyl phosphates, alkyl ether phosphates, alkylallyl ether phosphates and polycarboxylates, polyoxyethylene alkyl ethers such as polyoxyethylene stearyl ether, polyoxyethylene alkyl allyl ethers such as polyoxyethylene nonylphenyl ether, condensed polyoxyethylene alkyl allyl ethers of formaldehyde, block polymers using polyoxypropylene as lipophilic group, polyoxyethylene ethers of glycerol esters, sorbitan esters such as sorbitan monooleate, polyoxyethylene ethers of sorbitan esters, polyoxyethylene ethers of sorbitol esters, polyethylene glycol fatty acid esters, glycerol esters, polyglycerol esters, propylene glycol esters, sucrose esters, fatty acid alkanolamides, polyoxyethylene fatty acid amides, polyoxyethylene alkylamines and amine oxides.

Preferably, said nonionic dispersant is a linear or nonlinear ethoxylated fatty alcohol, more preferentially is a nonlinear ethoxylated fatty alcohol, more preferentially is an ethoxylated phenol.

Preferably, when the fatty alcohol is linear, it comprises at least 16 carbon atoms, and more preferentially it comprises at least 16 carbon atoms and is unsaturated.

Preferably, the dispersant is Ensoline® PHE LP from Arkema.

Preferably, said dispersant represents between 0.1% and 20% by weight of the composition, preferably between 0.5% and 20% by weight of the composition, preferably between 0.5% and 10% by weight of the composition, more preferentially between 1% and 5% by weight of the composition.

The composition according to the invention further comprises at least one thickener.

“Thickener” is understood to mean a compound capable of increasing the viscosity of a liquid phase.

The thickener may be organic or inorganic.

Preferably, the organic thickener is a polymer, preferably chosen from the group consisting of carboxylic acid homopolymers, copolymers and terpolymers, functionalized cellulose, such as cellulose ethers and esters, carboxymethylcellulose, functionalized carboxymethylcellulose, polysaccharides, preferably chosen from the group consisting of xanthan gum, guar gums, carob gum, carrageenans, alginates, locust bean gum, gellan gum, pectin, carrageenans, agar gum, preferably chosen from the group consisting of xanthan gum, guar gum, carob gum, carrageenans, and alginates, polyacrylates, polymethacrylates, functionalized polystyrene (SMA polymers), alpha-methylstyrene polymaleic acids, functionalized hydroxyethylcellulose esters, and functionalized and/or halogenated polyolefins.

Preferably, the inorganic thickener is chosen from the group consisting of bentonite, hectorite, silica, kaolinite, montmorillonite, attapulgite, and mixtures thereof.

Preferentially, the thickener is a polysaccharide, preferably chosen from the group consisting of xanthan gum, guar gum, carob gum, carrageenans, alginates, and mixtures thereof.

Particularly advantageously, the thickener is xanthan gum.

Preferably, said thickener represents between 0.05% and 20% by weight of the composition, preferably between 0.1% and 10% by weight of the composition, more preferentially between 0.3% and 5% by weight of the composition.

Preferably, at least 50%, preferably at least 60%, more preferentially 70%, more preferentially 80% and particularly preferably 90% by weight of the solid organic peroxide particles in the composition have a size of less than 500 micrometers. Preferably, at least 50% by weight of the solid organic peroxide particles in the composition have a size of greater than or equal to 100 micrometers and strictly less than 500 micrometers (the value of 500 micrometers is therefore excluded).

The size of the particles of the composition may be measured via various measurement techniques, notably (dynamic and static) light scattering techniques, measurements by rate of sedimentation, and microscopy. Preferably, the particle size of the composition according to the invention is measured by dry sieving, which consists in measuring the weight of material that passes through calibrated meshes of a sieve cloth. The sieves are superposed by decreasing mesh and the weight of material retained on each sieve is measured. This operation can is carried out by vibrating the whole sieve column using a VE 1000 sieve shaker from Retsch, operating for 20 minutes at an oscillation angle of 1.5 mm.

Preferably, the composition according to the invention comprises water.

Preferably, the composition according to the present invention comprises water so as to form the remainder of the composition (up to 100%). Preferably, the water is deionized water or distilled water.

Preferably, the composition according to the invention comprises less than 30% by weight of water, preferably less than 25% by weight of water, more preferentially less than 20% by weight of water, relative to the total weight of the composition.

Preferably, the composition according to the invention has a viscosity of between 2000 and 30 000 mPa·s (millipascal second), preferably of between 5000 and 15 000 mPa·s, measured at 25° C. with a Brookfield viscometer equipped with a Helipath T-E spindle rotating at a speed of 20 RPM.

The composition may also comprise at least one mineral filler, preferably a silica.

Preferably, the composition according to the present invention has a stability of at least 6 months at 20° C., preferably of at least 9 months at 20° C., more preferentially of at least 12 months at 20° C.

For the purposes of the present invention, “stability” is understood to mean the storage life, i.e. the time that elapses between the preparation of the composition and the first traces of phase separation that is expressed by the creaming of water or the decantation of the solid organic peroxide.

The separation can be determined visually.

### Process for Preparing the Composition

The invention also relates to a process for preparing a solid organic peroxide composition, comprising a step of mixing at least one solid organic peroxide, at least one polyol having a melting temperature of less than 40° C., preferably less than 30° C., more preferentially less than 20° C., at least one dispersant and at least one thickener.

Preferably, the process for preparing the composition is carried out at ambient temperature.

Preferably, said at least one solid organic peroxide is as defined above.

Preferably, said at least one polyol having a melting temperature of less than 40° C., preferably less than 30° C., more preferentially less than 20° C., is as defined above.

Preferably, said at least one nonionic dispersant is as defined above.

Preferably, said at least one thickener is as defined above.

Preferably, the process according to the invention is a process for preparing a phlegmatized organic peroxide. The phlegmatized organic peroxide thus obtained is stable during storage and handling.

The mixing step may for example be carried out using a rotor/stator device such as an Ultra Turrax®, colloid mills, pearl mills, ball mills, Dispax reactor, ultrasound mills or by a dispersion device equipped with butterfly rotors and/or Cowles blade disperser.

### Use

The invention also relates to the use of the composition as defined above as a polymerization initiator, in particular as a polymerization initiator of acrylic resins or of unsaturated polyester resins, preferably of acrylic resins, or as a polymer modifier, for example as a polymer crosslinking agent, grafting agent or rheology modifier, or as a bleaching agent, in particular as a laundry bleaching agent.

Preferably, the invention relates to the use of the composition as defined above as a polymerization initiator of acrylic resins or of unsaturated polyester resins.

The invention also relates to a process for polymerizing a polymerizable composition, comprising a step of bringing said polymerizable composition into contact with a composition according to the invention.

The polymerization step is in accordance with the polymerization steps of the prior art, which are well known to those skilled in the art.

The present invention also relates to the resin that is able to be obtained with said process.

The invention also relates to a process for modifying polymers, for example for crosslinking polymers, for grafting or modifying rheology, comprising a step of bringing said polymer and the composition according to the present invention into contact.

Said modification process is in accordance with the modification processes of the prior art, which are well known to those skilled in the art.

The present invention also relates to the polymer that is able to be obtained with the modification process according to the present invention.

The present invention also relates to a process for bleaching a product, in particular laundry, comprising a step of bringing said product into contact with a composition according to the invention.

The following examples serve to illustrate the invention without, however, exhibiting a limiting nature.

### EXAMPLES

### A. Preparation of a Solid Organic Peroxide Composition

Glycerol (GLICERINA 99.5 VEG. PH EUR EP 212 from Oqema Spa) and then the dispersant (Ensoline® PHE LP from Arkema (HLB=6.3)) are added into a 250 ml plastic beaker. The organic peroxide is then added, and then mixing is performed for 10 minutes using an IKA mixer equipped with an R 1381 3-bladed propeller stirrer (stirrer diameter 45 mm, shaft diameter 8 mm, shaft length 350 mm) at a speed of 1000 RPM. Lastly, the thickener (xanthan gum, XANTHAN GUM FCC/USP PH.EUR from Jungbunzlauer Austria AG) is introduced into the composition, which is mixed again at 1000 RPM for 5 minutes. The test is performed at ambient temperature (23±2° C.). The contents used are specified in the table below:

TABLE 1

| | Compo- | | Compo- | | Compo- | | Compo- | |
|---|---|---|---|---|---|---|---|---|
| | sition 1 | | sition 2 | | sition 3 | | sition 4 | |
| | % by | Mass | % by | Mass | % by | Mass | % by | Mass |
| | weight | (g) | weight | (g) | weight | (g) | weight | (g) |
| Gly- | 41% | 41 | 41.5% | 41.5 | 43.9% | 43.9 | 43.4% | 43.4 |
| cerol | | | | | | | | |
| Enso- | 2.5% | 2.5 | 2.5% | 2.5 | 0.1% | 0.1 | 0.1% | 0.1 |
| line ® | | | | | | | | |
| PHE | | | | | | | | |
| LP | | | | | | | | |
| Luper- | 56.0% | 56.0 | 56.0% | 56.0 | 56% | 56.0 | 56% | 56.0 |
| ox ® | | | | | | | | |
| A75 E | | | | | | | | |
| (ben- | | | | | | | | |
| zoyl | | | | | | | | |
| per- | | | | | | | | |
| oxide | | | | | | | | |
| at 75% | | | | | | | | |
| in | | | | | | | | |
| water) | | | | | | | | |
| Xan- | 0.5% | 0.5 | 0% | 0 | 0% | 0 | 0.5% | 0.5 |
| nthan | | | | | | | | |
| gum | | | | | | | | |
| TOTAL | 100.0% | 100.0 | 100.0% | 100.0 | 100.0% | 100.0 | 100.0% | 100.0 |

### B. Particle Size Distribution

2 g of sample obtained previously is placed on a 500 μm nylon cloth, itself placed above a 250 ml beaker. The sample is washed with approx. 50 ml of ethanol. The filter is inspected visually. It is noted whether no particles have remained on the filter or whether one or more particles remain on the filter.

### C. Appearance of the Composition

The appearance of the composition obtained in step A is analyzed visually.

### D. Results

The results obtained are presented in the table below:

TABLE 2

| | Presence of | | | |
|---|---|---|---|---|
| | organic | | Appearance | Appearance |
| | peroxide | | after 1 month of | after 12 months |
| | particles | | storage at | of storage at |
| | having a | Appearance | ambient | ambient |
| | diameter >500 | at the end of | temperature | temperature |
| Composition | μm | mixing | (23 ± 2° C.) | (23 ± 2° C.) |
| 1 | no | Homogeneous | Homogeneous | Homogeneous |
| 2 | no | Homogeneous | Heterogenous | Heterogenous |
| | | | (presence of a | (abundant |
| | | | semi- | presence of a |
| | | | transparent | semi- |
| | | | supernatant | transparent |
| | | | liquid at the | supernatant |
| | | | surface) | liquid at the |
| | | | | surface) |
| 3 | yes | Heterogenous | Heterogenous | Heterogenous |
| | | (presence of | (presence of | (presence of |
| | | visible grains) | visible grains | visible grains |
| | | | and a semi- | and a semi- |
| | | | transparent | transparent |
| | | | liquid in the | liquid in the |
| | | | bottom of the | bottom of the |
| | | | beaker) | beaker) |
| 4 | yes | Heterogenous | Heterogenous | Heterogenous |
| | | (presence of | (presence of | (presence of |
| | | visible grains) | visible grains) | visible grains) |

### E. Conclusion

It is observed that the use of a thickener in combination with a dispersant makes it possible to obtain a composition that is very stable over a prolonged storage period.

Moreover, it is noted that an insufficient quantity of dispersant does not make it possible to obtain a homogeneous mixture.

1. A composition comprising:
10% to 70% by weight, relative to the total weight of the composition, of at least one solid organic peroxide,
at least one polyol having a melting temperature of less than 40° C.,
from 0.5% to 20% by weight, relative to the total weight of the composition, of at least one dispersant, and
at least one thickener.

2. The composition as claimed in claim 1, in which the organic peroxide is chosen from the group consisting of solid diacyl peroxides, solid peroxydicarbonates, solid ketone peroxides, solid peroxy esters, solid hydroperoxides, solid dialkyl peroxides, and mixtures thereof.

3. The composition as claimed in claim 1, in which the solid organic peroxide is present in a content ranging from 20% to 60% by weight, relative to the total weight of the composition.

4. The composition as claimed in claim 1, in which said polyol is chosen from the group consisting of glycerol or diglycerol, and mixtures thereof.

5. The composition as claimed in claim 1, in which said polyol is present in a content ranging from 5% to 60% by weight of the composition.

6. The composition as claimed in claim 1, in which the dispersant has an HLB of less than 14.

7. The composition as claimed in claim 1, in which said dispersant is a nonionic dispersant.

8. The composition as claimed in claim 1, in which said dispersant represents between 0.5% and 10% by weight of the composition.

9. The composition as claimed in claim 1, in which said thickener is a polysaccharide.

10. The composition as claimed in claim 1, in which said thickener represents between 0.05% to 20% by weight of the composition.

11. The composition as claimed in claim 1, said composition having has a stability of at least 6 months at 20° C.

12. The composition as claimed in claim 1, in which at least 50%, by weight of the particles have a size of less than 500 micrometers.

13. A method of using the composition as defined in claim 1 as a polymerization initiator or as a bleaching agent.

14. A process for preparing a solid organic peroxide composition, comprising a step of mixing at least one solid organic peroxide, at least one polyol having a melting temperature of less than 40° C., at least one dispersant, and at least one thickener.
